(19) United States
(12) Patent Application Publication
(10) Pub. No.: US 2005/0031622 A1
(43) Pub. Date: Feb. 10, 2005

(54) Treatment and diagnosis of cancer using inositolphosphoglycans antagonists

(75) Inventors: Thomas William Rademacher, Oxford (GB); Hugo Caro, London (GB)

Correspondence Address: QUINE INTELLECTUAL PROPERTY LAW GROUP, P.C., P O BOX 458, ALAMEDA, CA, 94501, US

(73) Assignee: Rodaris Pharmaceuticals Limited

(21) Appl. No.: 10/870,562

(22) Filed: Jun. 16, 2004

Related U.S. Application Data
(63) Continuation of application No. 09/868,968, filed on Jun. 22, 2001, now abandoned.

(30) Foreign Application Priority Data
Dec. 23, 1998 (GB) 9828564.6

(51) Int. Cl. A61K 39/395

(52) U.S. Cl. 424/155.1

(57) ABSTRACT

Inositolphosphoglycans (IPGs), and in particular A-type substances comprising myo-inositol, are tumour autocrine factors (TAFs), that is factors which cause tumour cell proliferation. The use of A-type IPG antagonists for the treatment of cancer and a method for the diagnosis or prognosis of cancer based on the presence or amount of IPGs in a sample from a patient is disclosed.

### FIELD OF THE INVENTION

The present invention relates to materials and methods for the treatment and diagnosis of cancer.

### BACKGROUND OF THE INVENTION

It has been observed that when tumour cells are grown in culture, the rate of proliferation of the cells depends on the cell density in the culture. It has been hypothesised that this increase in the rate of proliferation as the number of cells increases is due to the presence of tumour autocrine factors (TAFs) in the cell supernatant. TAFs are believed to be growth factors produced by the tumour cells which cause them to proliferate in the absence of external stimuli such as hormones.

There is some experimental evidence supporting the existence of TAFs in the culture supernatant. If the supernatant from a high density tumour cell culture is applied to a dilute culture, the rate of proliferation of the cells in the dilute culture quickly accelerates to the level associated with cells in a high density culture. It has also been observed that if the supernatant is boiled prior to addition to the dilute culture, the acceleration in the proliferation rate of the cells is not observed.

Witter et al (1987) have reported that conditioned medium from Reuber H-35 or Fao hepatoma cells contained autocrine factors that both stimulated DNA synthesis and activated acetyl-coenzyme A (CoA) carboxylase in serum deprived cells. They also found that the factors increased the cell number and mitotic index in tumour cell cultures. The investigators fractionated conditioned medium from H-35 hepatoma cells and found that two low molecular weight components (MW<1000) co-purified with the activity of increasing DNA synthesis. Radiolabelling incorporation studies suggested the possible incorporation of glucosamine and galactose, but no incorporation of myo-inositol or mannose. However, the authors were unable to purify, isolate or characterise the autocrine factor from conditioned medium and do not provide any definite suggestions as to the structure or activity of their autocrine factor.

### SUMMARY OF THE INVENTION

Broadly, the present invention is based on the isolation and identification of TAFs, and in particular on the finding that TAFs are inositolphosphoglycans (IPGs) such as A or P-type IPGs. In particular, the results show that the TAFs include myo-inositol and by extension A-type IPGs.

Accordingly, in a first aspect, the present invention provides the use of a substance which is an IPG antagonist for the preparation of a medicament for the treatment of cancer. Preferably, the IPG antagonist has the property of reducing tumour cell proliferation.

In the present application, “IPG antagonists” includes substances which have one or more of the following properties:
(a) substances which are capable of inhibiting release of IPGs, e.g. inhibitors of enzymes which cause their release such as glycosylphosphatidylinositol specific phospolipases (GPI-PL);
(b) substances which are capable of reducing the levels of IPGs by binding to them, e.g. anti-IPG antibodies, and preferably neutralising one or more IPG biological activities, more especially the activity of IPGs causing tumour cell proliferation; and/or,
(c) substances which are capable of reducing the effects of IPGs, e.g. competitive antagonists such as IPGs which are biologically inactive in the species in which they are used, e.g. the use of porcine liver A-type IPGs in humans.

In preferred embodiments, the IPG antagonist is an antibody capable of specifically binding IPGs and preferably which does not cross-react with the common reactive determinant of GPI anchored proteins. Preferably, the antibody is a monoclonal antibody, examples of which are disclosed below.

In other embodiments, the antagonist inhibits the release of IPGs. This suggests that an enzyme responsible for the release of the IPG TAFs is a GPI-PLC, which has not been previously identified in mammalian tissues. Therefore, inhibitors of GPI-PLC may be used as IPG antagonists for treatment of cancer.

In a further aspect, the present invention provides a method for the diagnosis or prognosis of cancer, the method comprising determining the presence or amount of IPGs in a sample from a patient. The presence or amount of IPGs in a given sample is typically then compared to standards from healthy and cancerous tissues.

Preferably, the presence or amount of the IPGs is determined by measuring a characteristic activity of an A or P-type IPG, details of which are set out below. Alternatively, the presence or amount of a characteristic component of IPGs and in particular IPG TAFS, could be used as a diagnostic marker. Other components of IPGs that might be used as diagnostic markers are set out below.

In a further aspect, the present invention provides the use of cellulose column chromatography in the purification of IPGs.

In a further aspect, the present invention provides a method of purifying or isolating IPGs, the method comprising making contacting a sample containing IPGs with a column containing cellulose, and eluting the IPGs from the column. Preferred conditions preparing the column and eluting IPG containing fractions are set out below.

Embodiments of the present invention will now be described by way of example and not by limitation with reference to the accompanying figures.

### BRIEF DESCRIPTION OF THE FIGURES

FIG. 1 shows the fractionation of conditioned medium by cellulose column chromatography. The results are presented as counts per minute (cpm) per fraction. The arrows indicate the solvent system used at a particular fraction.

FIG. 2 shows the effects on DNA synthesis of conditioned medium. Conditioned medium at two different concentrations (10% and 20%) was assayed for stimulating activity in test Fao cells. The results are presented as radioactivity (cpm) obtained after incorporation of ³H-thymidine into DNA, for control (no serum addition), FCS (5% foetal calf serum+5% foetal bovine serum addition), and the fractions obtained. The arrows indicate the eluent system used at a particular fraction.

FIG. 3 shows phosphate analysis of conditioned medium fractionated by cellulose column chromatography. Results are presented as OD obtained per fraction. Arrows indicate the eluent system used at a particular fraction.

FIG. 4 shows the effect on the proliferation of FaO cells of conditioned medium treated with anti-IPG monoclonal antibodies. Results are presented as radioactivity (cpm) obtained after incorporation of ³H-thymidine into DNA, for control (no serum addition), FCS (5% foetal calf serum+5% foetal bovine serum addition), and samples containing the antibodies.

FIGS. 5 and 6 show rat liver IPGs A and P fractionation profiles. The results are shown as radioactivity (cpm) obtained after incorporation of ³H-thymidine into DNA of 3T3EFGTR17 cells, for control (no serum addition), FCS (10% foetal calf addition), and fractions. All fractions were assayed at a final IPG-Aconcentration of 1/80, except for fraction 26 of IPG-P (FIG. 5) which was also tested at a final concentration of 1/40 and 1/160. All fractions of the IPG-A (FIG. 6) were tested at a final concentration of 1/80. The arrows indicate the eluent system used at a particular fraction.

FIG. 7 shows the phosphate analysis of rat liver IPG-P fractionated from rat liver by cellulose column chromatography. Results are presented as OD obtained per fraction. Arrows indicate the eluent system used at a particular fraction.

### DETAILED DESCRIPTION OF THE INVENTION

IPGs

Studies have shown that A-type mediators modulate the activity of a number of insulin-dependent enzymes such as cAMP dependent protein kinase (inhibits), adenylate cyclase (inhibits) and cAMP phospho-diesterases (stimulates). In contrast, P-type mediators modulate the activity of insulin-dependent enzymes such as pyruvate dehydrogenase phosphatase (stimulates), glycogen synthase phosphatase (stimulates) and cAMP dependent protein kinase (inhibits). The A-type mediators mimic the lipogenic activity of insulin on adipocytes, whereas the P-type mediators mimic the glycogenic activity of insulin on muscle. Both A- and P-type mediators are mitogenic when added to fibroblasts in serum free media. The ability of the mediators to stimulate fibroblast proliferation is enhanced if the cells are transfected with the EGF-receptor. A-type mediators can stimulate cell proliferation in the chick cochleovestibular ganglia.

Soluble IPG fractions having A-type and P-type activity have been obtained from a variety of animal tissues including rat tissues (liver, kidney, muscle brain, adipose, heart) and bovine liver. A- and P-type IPG biological activity has also been detected in human liver and placenta, RBC infected with Plasmodium yoelii and mycobacteria. The ability of an anti-inositolglycan antibody to inhibit insulin action on human placental cytotrophoblasts and BC3H1 myocytes or bovine-derived IPG action on rat diaphragm and chick cochleovestibular ganglia suggests cross-species conservation of many structural features.

A-type substances are cyclitol-containing carbohydrates, also containing Zn²⁺ ion and optionally phosphate and having the properties of regulating lipogenic activity and inhibiting cAMP dependent protein kinase. They may also inhibit adenylate cyclase, be mitogenic when added to EGF-transfected fibroblasts in serum free medium, and stimulate lipogenesis in adipocytes.

P-type substances are cyclitol-containing carbohydrates, also containing Mn²⁺ and/or Zn²⁺ ions and optionally phosphate and having the properties of regulating glycogen metabolism and activating pyruvate dehydrogenase phosphatase. They may also stimulate the activity of glycogen synthase phosphatase, be mitogenic when added to fibroblasts in serum free medium, and stimulate pyruvate dehydrogenase phosphatase.

Methods for obtaining A-type and P-type IPGs are set out in Caro et al, 1997, and in WO98/11116 and WO98/11117. In summary, the methods disclosed in these applications involve:
(a) making an extract by heat and acid treatment of IPG source material;
(b) after centrifugation and charcoal treatment, allowing the resulting solution to interact overnight with an AG1-X8 (formate form) anion exchange resin;
(c) collecting a fraction having A-type IPG activity obtained by eluting the column with 50 mM HCl and/or collecting a fraction having P-type IPG activity obtained by eluting the column with 10 mM HCl;
(d) adjusting to pH 4 (not to exceed pH 7.8) and lyophilising the fraction to isolate the substance;
(e) employing descending paper chromatography using 4/1/1 butanol/ethanol/water as solvent;
(f) purification using high-voltage paper electrophoresis in pyridine/acetic acid/water; and,
(g) purification using Dionex anion exchange chromatography or purification and isolation using Vydac HPLC chromatography to obtain the isolated IPG.

As disclosed herein, it is also possible to employ column chromatography using cellulose, and especially microcrystalline cellulose, in the isolation or purification of IPGs. Exemplary conditions are provided in the experimental section below.

Antagonists

In the present invention, “IPG antagonists” includes substances which have one or more of the following properties:
(a) substances which are capable of inhibiting release of IPGs, e.g. inhibitors of enzymes which cause their release such as GPI-PL;
(b) substances which are capable of reducing the levels of IPGs by binding to them and preferably neutralising an IPG biological activity; and/or,
(c) substances which are capable of reducing the effects of IPGs, e.g. competitive antagonists such as IPGs which are biologically inactive in the species in which they are used, e.g. the use of A-type IPGs as obtainable from porcine liver in humans.

Under (b) and (c), preferably the biological activity of the IPGs which is affected by the antagonists is the tumour cell proliferation caused by the IPGs when they are added to cell cultures. This can be readily determined using the assays described in the examples or other techniques well known to those skilled in the art.

Examples of IPG antagonists of type (b) include specific binding proteins, e.g. naturally occurring specific binding proteins that can be obtained by screening biological materials for substances that bind to IPGs.

In a further example, the antagonists are antibodies capable of specifically binding to IPGs. The production of polyclonal and monoclonal antibodies is well established in the art. Examples of anti-IPG monoclonal antibodies are produced by hybridoma cell lines 2F7, 2D1 and 5H6 deposited at European Collection of Cell Cultures (ECACC) under accession numbers 98051201, 98031212 and 98030901 on 12 May 1998 ('201) and 9 Mar. 1998 ('212 and '901).

Monoclonal antibodies are particularly useful as they can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP 0 184 187 A, GB 2 188 638 A or EP 0 239 400 A. A hybridoma producing a monoclonal antibody may be subject to genetic mutation or other changes, which may or may not alter the binding specificity of antibodies produced.

Antibodies may be obtained using techniques which are standard in the art. Methods of producing antibodies include immunising a mammal (e.g. mouse, rat, rabbit, horse, goat, sheep or monkey) with an immunogen, e.g. an IPG or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al, Nature, 357:80-82, 1992). Isolation of antibodies and/or antibody-producing cells from an animal may be accompanied by a step of sacrificing the animal.

As an alternative or supplement to immunising a mammal with an IPG, an antibody specific for the IPG may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies according to the present invention may be modified in a number of ways. Indeed the term “antibody” should be construed as covering any binding substance having a binding domain with the required specificity. Thus, antagonist antibodies includes antibody fragments, derivatives, functional equivalents and homologues of antibodies, including synthetic molecules and molecules whose shape mimics that of an antibody enabling it to bind an antigen or epitope.

Examples of antibody fragments which capable of binding an IPG or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Humanised antibodies in which CDRs from a non-human source are grafted onto human framework regions, typically with the alteration of some of the framework amino acid residues, to provide antibodies which are less immunogenic than the parent non-human antibodies, are also included within the present invention and can be produced by the skilled person using techniques well known in the art.

A hybridoma producing a monoclonal antibody according to the present invention may be subject to genetic mutation or other changes. It will further be understood by those skilled in the art that a monoclonal antibody can be subjected to the techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB-A-2188638 or EP-A-0239400. Cloning and expression of chimeric antibodies are described in EP-A-0120694 and EP-A-0125023.

Hybridomas capable of producing antibody with desired binding characteristics are within the scope of the present invention, as are host cells, eukaryotic or prokaryotic, containing nucleic acid encoding antibodies (including antibody fragments) and capable of their expression. The invention also provides methods of production of the antibodies including growing a cell capable of producing the antibody under conditions in which the antibody is produced, and preferably secreted.

The antibodies described above may also be employed in the diagnostic aspects of the invention by tagging them with a label or reporter molecule which can directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule.

One favoured mode is by covalent linkage of each antibody with an individual fluorochrome, phosphor or laser dye with spectrally isolated absorption or emission characteristics. Suitable fluorochromes include fluorescein, rhodamine, phycoerythrin and Texas Red. Suitable chromogenic dyes include diaminobenzidine.

Other reporters include macromolecular colloidal particles or particulate material such as latex beads that are coloured, magnetic or paramagnetic, and biologically or chemically active agents that can directly or indirectly cause detectable signals to be visually observed, electronically detected or otherwise recorded. These molecules may be enzymes which catalyse reactions that develop or change colours or cause changes in electrical properties, for example. They may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed.

In a further embodiment, the IPG antagonists are competitive antagonists, such as synthetic compounds or IPGs which are biologically inactive in the relevant respect, e.g IPGs which do not have a TAF activity such as enhancing the proliferation of tumour cells. An example of this latter type of antagonist is the use of porcine liver A-type IPGs in humans as described in GB-A-9828560.4 and related applications. Synthetic compounds may be produced by chemical techniques or using combinatorial chemistry, and then screened for IPG antagonist activity. These compounds may be useful in themselves or may be used in the design of mimetics, providing candidate lead compounds for development as pharmaceuticals. Synthetic compounds might be desirable where they are comparatively easy to synthesize or where they have properties that make them suitable for administration as pharmaceuticals, e.g. antagonist which are peptides may be unsuitable active agents for oral compositions if they are degraded by proteases in the alimentary canal. Mimetic design, synthesis and testing is generally used to avoid randomly screening large number of molecules for a target property.

Pharmaceutical Compositions

IPGs or IPG antagonists can be formulated in pharmaceutical compositions. These compositions may comprise, in addition to one or more of IPGs or antagonists, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilisers, buffers, antioxidants and/or other additives may be included, as required.

Whether it is a polypeptide, antibody, peptide, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Oslo, A. (ed), 1980.

The compositions may be administered alone or in combination with other treatments, either simultaneously or sequentially dependent upon the condition to be treated. Thus, in the treatment of cancer, the IPG antagonists can be administered in combination with other chemotherapy or radiotherapy.

Diagnostic Methods

Methods for determining the concentration of analytes in samples from individuals are well known in the art and readily adapted by the skilled person in the context of the present invention to determine whether an individual has an elevated level of IPGs, and so has or is at risk of cancer. The purpose of such analysis may be used for diagnosis or prognosis to assist a physician in determining the severity or likely course of the cancer and/or to optimise treatment of it. Examples of diagnostic methods are described in the experimental section below.

Preferred diagnostic methods rely on the detection of IPGS, employing biological samples such as blood, serum, urine or tissue samples (especially from a suspected tumour site).

The assay methods for determining the concentration of IPGs typically employ a binding agent having binding sites capable of specifically binding to the IPGs in preference to other molecules. Examples of binding agents include antibodies (examples of which are provided above), receptors and other molecules capable of specifically binding IPGs. Conveniently, the binding agent is immobilised on solid support, e.g. at a defined location, to make it easy to manipulate during the assay.

The sample is generally contacted with a binding agent under appropriate conditions so that IPGs present in the sample can bind to the binding agent.

The fractional occupancy of the binding sites of the binding agent can then be determined either by directly or indirectly labelling the analyte or by using a developing agent or agents to arrive at an indication of the presence or amount of the analyte in the sample. Typically, the developing agents are directly or indirectly labelled (e.g. with radioactive, fluorescent or enzyme labels, such as horseradish peroxidase) so that they can be detected using techniques well known in the art. Directly labelled developing agents have a label associated with or coupled to the agent. Indirectly labelled developing agents may be capable of binding to a labelled species (e.g. a labelled antibody capable of binding to the developing agent) or may act on a further species to produce a detectable result. Thus, radioactive labels can be detected using a scintillation counter or other radiation counting device, fluorescent labels using a laser and confocal microscope, and enzyme labels by the action of an enzyme label on a substrate, typically to produce a colour change. In further embodiments, the developing agent or analyte is tagged to allow its detection, e.g. linked to a nucleotide sequence which can be amplified in a PCR reaction to detect the analyte. Other labels are known to those skilled in the art are discussed below. The developing agent(s) can be used in a competitive method in which the developing agent competes with the analyte for occupied binding sites of the binding agent, or non-competitive method, in which the labelled developing agent binds analyte bound by the binding agent or to occupied binding sites. Both methods provide an indication of the number of the binding sites occupied by the analyte, and hence the concentration of the analyte in the sample, e.g. by comparison with standards obtained using samples containing known concentrations of the analyte.

The sample is generally contacted with the binding agent under appropriate conditions which allow the analyte in the sample to bind to the binding agent.

There is also an increasing tendency in the diagnostic field towards miniaturisation of such assays, e.g. making use of binding agents (such as antibodies or nucleic acid sequences) immobilised in small, discrete locations (microspots) and/or as arrays on solid supports or on diagnostic chips. These approaches can be particularly valuable as they can provide great sensitivity (particularly through the use of fluorescent labelled reagents), require only very small amounts of biological sample from individuals being tested and allow a variety of separate assays can be carried out simultaneously. This latter advantage can be useful as it provides an assay employing a plurality of analytes to be carried out using a single sample. Examples of techniques enabling this miniaturised technology are provided in WO84/01031, WO88/1058, WO89/01157, WO93/8472, WO95/18376/WO95/18377, WO95/24649 and EP 0 373 203A. Thus, in a further aspect, the present invention provides a kit comprising a support or diagnostic chip having immobilised thereon a plurality of binding agents, at least one of which is capable of binding to an IPG as described above, optionally in combination with other reagents (such as labelled developing reagents) needed to carrying out an assay.

Materials and Methods

Anti-IPG Antibodies

Anti-IPG antibodies were produced by culturing hybridoma cell lines 2F7, 2D1 and 5H6 deposited at European Collection of Cell Cultures (ECACC) under accession numbers 98051201, 98031212 and 98030901, and isolating the anti-IPG antibodies thus produced.

Cellulose Purification of IPGs

Microcrystalline cellulose (Merck Avicel®) was fined by suspension in double-distilled water followed by standing for 10 minutes and decanting of the unsettled suspension. After 12 repeats, the settled material was shaken vigorously with two volumes of fresh water and poured into the top of a Pharmacia® glass column of 16 mm i.d. with the top plunger removed. Water was allowed to flow out under gravity at 3 ml/min. After allowing the solvent level to drop to the top of the packed bed, the plunger was replaced and water pumped through the column at 5 ml/min using a Pharmacia® peristaltic pump. The column was treated with the following solvents:

| | |
|---|---|
| Water | 150 ml |
| 50% aqueous methanol | 50 ml |
| Methanol | 50 ml |
| Butanol/methanol/water (4:1:1) | 150 ml |

All solvents were degassed prior to use by brief swirling under vacuum. After the final solvent, the plunger was removed, and the solvent level allowed to drop to the top of the packed bed under gravity, whereupon flow was stopped.

The sample (2 ml of conditioned cell medium or IPG extract from two rat livers) was swirled for 5 minutes with 2 ml of the cellulose suspension and the entire mixture lyophilised.

The dry cellulose material was suspended in 2 ml of butanol/ethanol/water (B:W:E) (4:1:1) and applied gently and evenly to the top of the packed bed. The remaining material was washed in using a further 2 ml of the solvent. The solvent level was again dropped to the bed level before commencing pumping with B:W:E (4:1:1) at 5 ml/min. Fractions were collected in glass vessels as follows, either by hand into flasks or using a Gilson model 204 fraction collector set to 1 minute/tube:

| | |
|---|---|
| Butanol/methanol/water (4:1:1) | 150 ml |
| Methanol | 50 ml |
| 50% aqueous methanol | 10 × 5 ml fractions |
| Water | 20 × 5 ml fractions |
| HCl(aq.) pH 1.3 | 10 × 5 ml fractions |
| HCl(aq.) pH 1.3 | 75 ml |

The samples were evaporated to dryness using a rotary evaporator or Speedvac at below 30° C. Samples were dissolved in 200 μl of water and stored at −20° C.

Preparation of Conditioned Medium (CM) from H4IIE Cells

(a) H4IIE cells were cultured on 75 cm² Falcon cap vented tissue culture flasks at cell density of 2-6×10⁴ cells/cm² using DMEM (Gibco Cat. No. 31885), supplemented at 5% with a 1:1 mixture of foetal calf serum and heat inactivated calf serum and incubate at 37° C., 5% CO₂ atmosphere until 100% confluence.

(b) At confluence (15-17×10⁶ cells/T75 flask), the cells were washed twice using 15-20 mL Hank's Balance Salt Solution pH 7.4 (HBSS, Gibco Cat No. 14174-053) and 5 ml Trypsin-EDTA solution (Sigma Cat. No. T 4171) was added. The excess of Trypsin was then removed, leaving in the flask only 2 ml of liquid which was then incubated for 10 min.

(c) When the cells detached, 8 ml of growth medium was added and the mixture was centrifuged for 5 min at 1000 rpm. The supernatant was discharged and the pellet resuspended by adding 10 ml growth medium. The cell suspension was transferred into a sterile 25 ml universal container, 10 ml of the same medium was added, the cap closed tightly and the container rocked several times to produce a homogeneous cell suspension.

(d) The cells were subcultured 1/10 or 1/20 (1-2.5×10⁴/cm²) in 75 cm² Falcon cap vented flask, using the same medium and incubation conditions described in step (a). When the cells seeded at 1/10 reached 70% confluence (11.5-12.0×10⁶ cells/T75 flask, normally 3 days after), the culture was washed three times with 20 ml serum free DMEM and then incubated for 3 hours. This procedure was repeated twice.

(e) The culture was incubated for 48 hr at 37° C., 5% CO₂ atmosphere, and then the conditioned medium was transferred into a centrifuge tube, and cell debris removed by centrifugation at 1000 g for 10 min at 4° C. and then at 105,000 g for 60 min at 4° C. The supernatant was sterilized by filtration and stored at −80° C.

Biosynthetic Labelling and Extraction of Conditioned Medium

(a) H4IIE cells grown to 50-60% confluence in T75 cap vented flask Falcon (Falcon Cat. No F3111) (8-9×10⁶ cells/T75 flask) in DMEM medium, supplemented with 5% foetal calf serum and 5% heat inactivated calf serum, were washed twice using HBSS at 37° C. 10 mL of fresh growth medium was added and then 250 mCi of the ³H-labelled precursors (Glucosamine, Galactose, Mannose and myo-Inositol). The culture was incubated at 37° C., 5% CO₂ atmosphere for 24 hours.

(b) After 24 hours, the cells were washed three times as described in step (d) above and the cells incubated and the medium harvested as in step (e).

(c) The supernatants obtained were adjusted to pH 3.0 with HCl (c), and prewashed activated charcoal (25 mg/mL) added and stirred at 4° C. for 30 min. The suspension was transferred into centrifuge tubes and the charcoal spun down by centrifugation at 20,000 g for 30 min at 4° C.

(d) The supernatant was sterilized using a 0.2 μm pore size filter and stored at 4° C. If it was not required for immediate use, it was freeze-dried and kept at −80° C. until needed.

Bioactivity Assay of H4IIE Conditioned Medium by Measuring the Proliferation of FaO Cells

(a) FaO cells grown at 60-70% confluence in T75 Falcon cap-vented tissue culture flasks Gibo Cat. No F3111 with RPMI-1640 (Gibco Cat. No. 21875), supplemented at 5% with a 1:1 mixture of foetal calf serum and heat inactivated calf serum were washed twice with 20 ml of HBSS pH 7.4 (Gibco Cat. No. 14174). The cells were detached with Trypsin-EDTA solution (Sigma Cat. No. T4171) as described in steps (b) and (c) of “Preparation of conditioned medium from H4IIE” protocol set out above.

(b) The cell suspension was counted twice and the required volume to dilute the suspension at a density of 2.5-3.0×10³ cell/ml was calculated. The calculated volume of growth medium was added and the cells were transferred into a sterile 25 ml universal container. The container was rocked gently several times to produce a homogenous cell suspension.

(c) The cells were plated in a 96-wells Falcon U bottom shape microwell plate by adding 100 ml of cell suspension per well and incubated at 37° C. in a 5% CO₂ atmosphere incubator. After 24 hours, each well was washed three times with 100 ml of serum free medium and incubates for a further 24 hours.

(d) The medium was replaced by adding serum free medium and the cells incubated for three additional hours. Meanwhile, the IPG samples or medium supplementation at different concentrations (ranging from 1% to 20.0%) of H4IIE conditioned medium were prepared. 100 μl serum free medium was added to the controls, 100 μl completed growth medium was added to the positive controls and 100 μl of medium supplemented with H4IIE conditioned medium.

(e) The cells were incubated at 37° C. in a 5% CO₂ atmosphere incubator for 18 hours and then 1 mCi ³H-thymidine was added to each well. After 4 hours incubation, the radioactive medium was removed and replaced with 50 μl trypsin-EDTA solution. The cells were incubated for 10 min and the DNA collected on filters (Helis Bio Ltd. Cat No 11731) using a cell harvester (SKATRON 12 well/cell harvester). The filters were transferred to scintillation vials, 2 ml of scintillation cocktail was added to each vial and counted for one minute in a Beckman scintillation counter.

Assays

Phosphate analysis and PDH activation assays were carried out as described in Caro et al, 1997.

Results

Fractionation of Conditioned Medium by Cellulose Column Chromatography

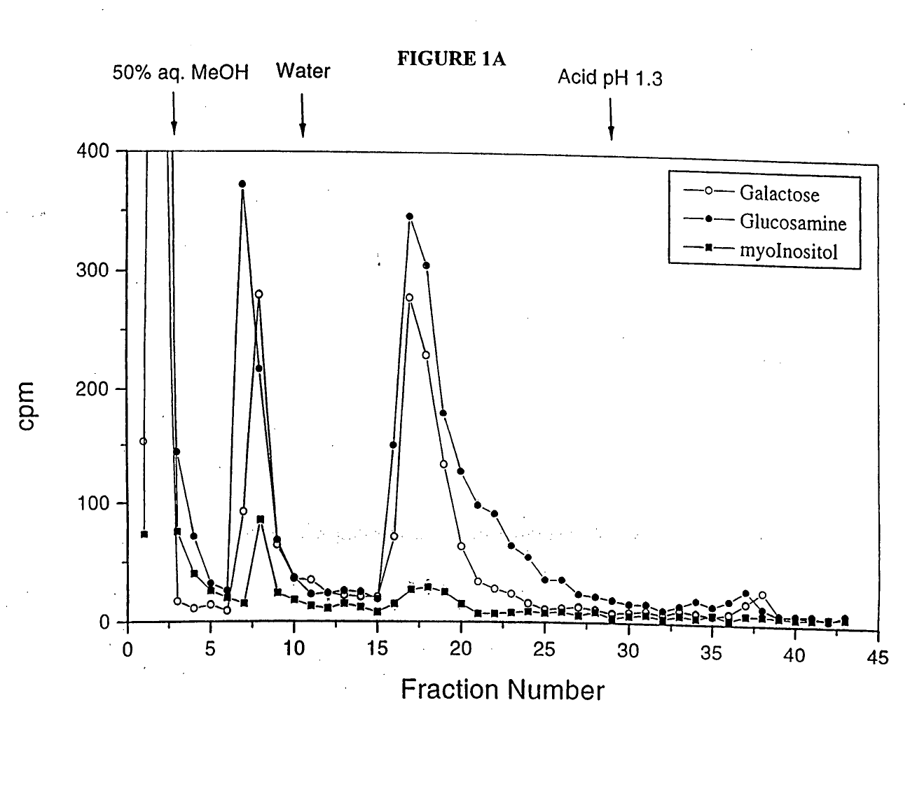

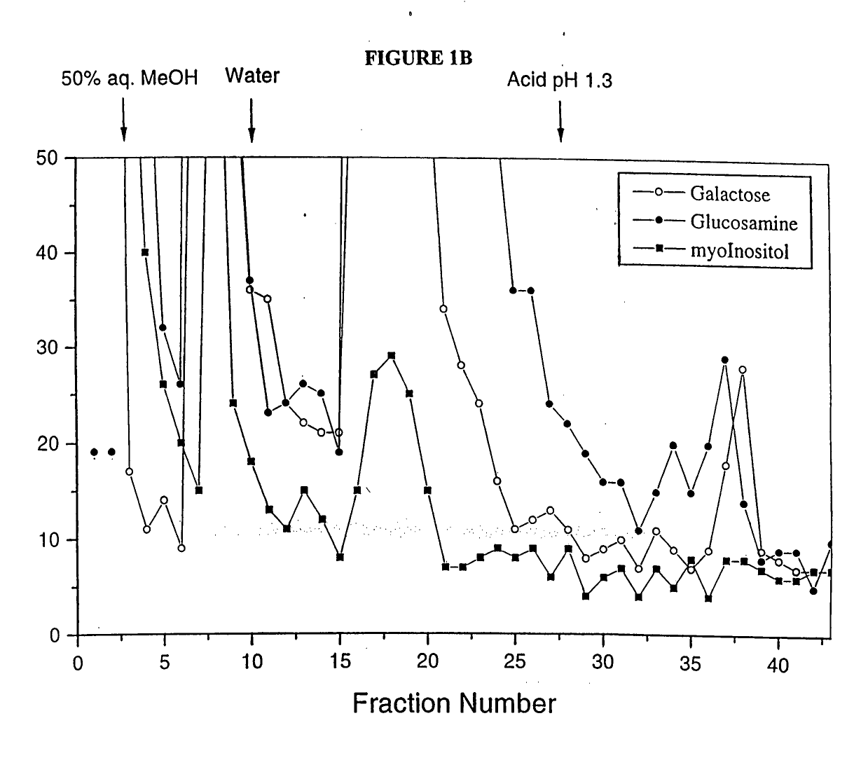

Conditioned medium biosyntheticaly labelled with ³H myo-inositol (squares), glucosamine (circles) or galactose (open circles) at 1.35E5, 2.85E5 and 2.50E5 cpm respectively, obtained as described in Methods, was separated by cellulose column chromatography, 5 ml fractions were collected and 200 μl counted in a Beckman liquid scintillation counter. The results are presented in FIG. 1 as counts per minute (cpm) per fraction. The arrows indicate the solvent system used at a particular fraction. The results in FIG. 1 show that these radioactive components are incorporated into TAF fractions obtained from the supernatant and that the TAF fractions can be isolated by the same procedures as IPGs.

Effects on DNA Synthesis of Conditioned Medium

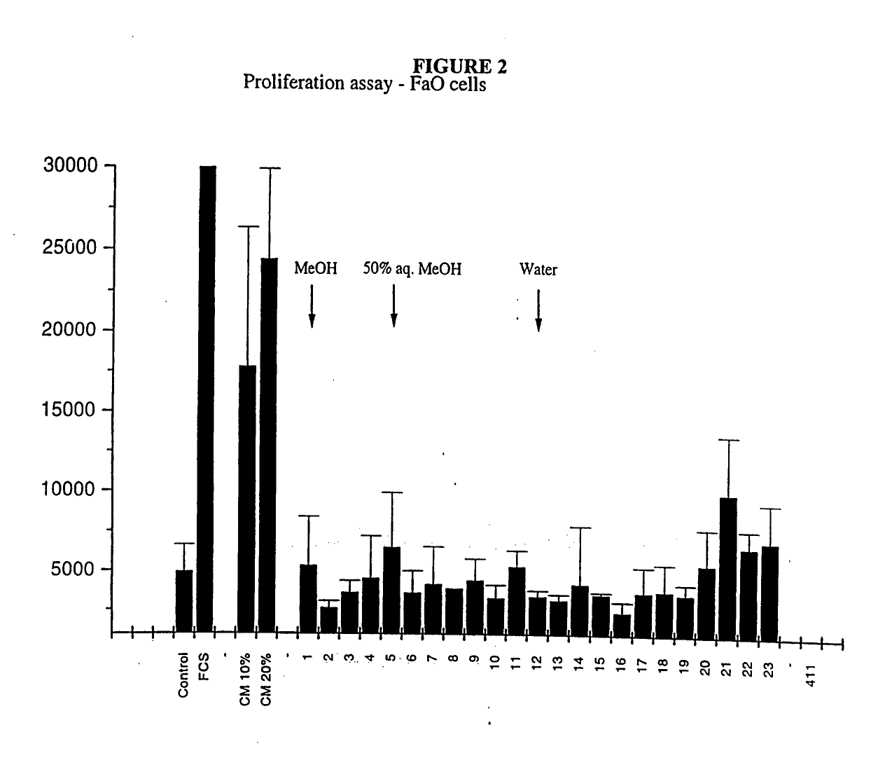

Conditioned medium at two different concentrations (10% and 20%) was assayed for stimulating activity in test Fao cells, as under Methods. The fractions obtained by purification of conditioned medium (2 ml) by cellulose column chromatography were concentrated to dryness, dissolved in water (200 μl), and also assessed for activity (4 μl). The results are presented in FIG. 2 as radioactivity (cpm) obtained after incorporation of ³H-thymidine into DNA, for control (no serum addition), FCS (5% foetal calf serum+5% foetal bovine serum addition), and the fractions obtained. The arrows indicate the eluent system used at a particular fraction. The results show that some of the fractions, and in particular fraction 21 causes proliferation of Fao cells as does the addition of conditioned medium.

Phosphate Analysis of Conditioned Medium

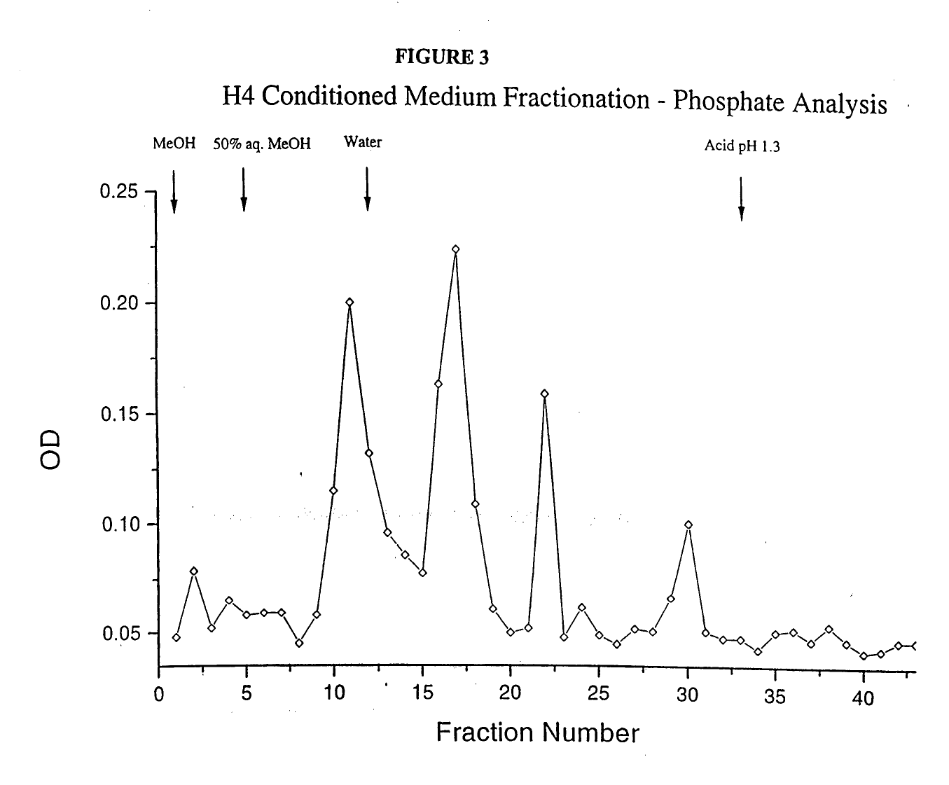

Conditioned medium (2 ml) was fractionated by column chromatography using cellulose as described under methods. Fractions (5 ml) were collected, concentrated to dryness, dissolved in water (200 μl and a portion (10 μl) was assayed for phosphate as described in Caro et al, 1997. Briefly, samples were evaporated to dryness and hydrolized with perchloric acid (70% by volume) at 180° C. for 30 min. After cooling at room temperature, distilled water (250 μl) was added. Ammonium molybdate and ascorbic acid were sequentially added, yielding final concentrations of 3 and 72 mM, respectively. Colour development was achieved by heating at 95° C. for 15 min. Optical absorbance was measured at 650 nm. The results are presented in FIG. 3 as OD obtained per fraction. Arrows indicate the eluent system used at a particular fraction. The results show that the fractions co-purifying with TAF activity contain phosphate, supporting the fact that TAFs are IPGs.

Effect on the Proliferation of FaO Cells of Conditioned Medium Treated with Anti-IPG Monoclonal Antibodies

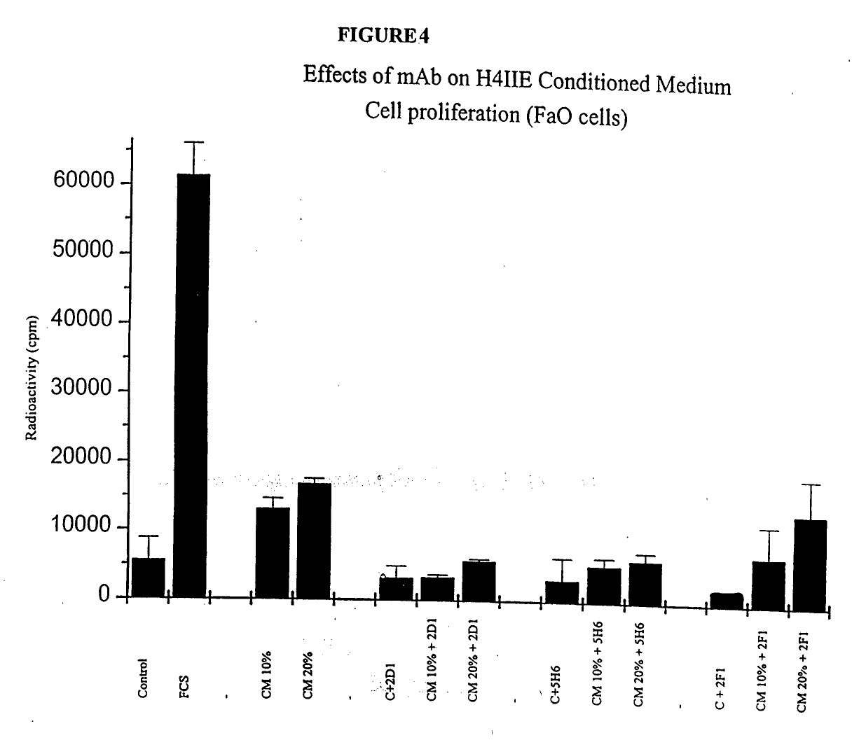

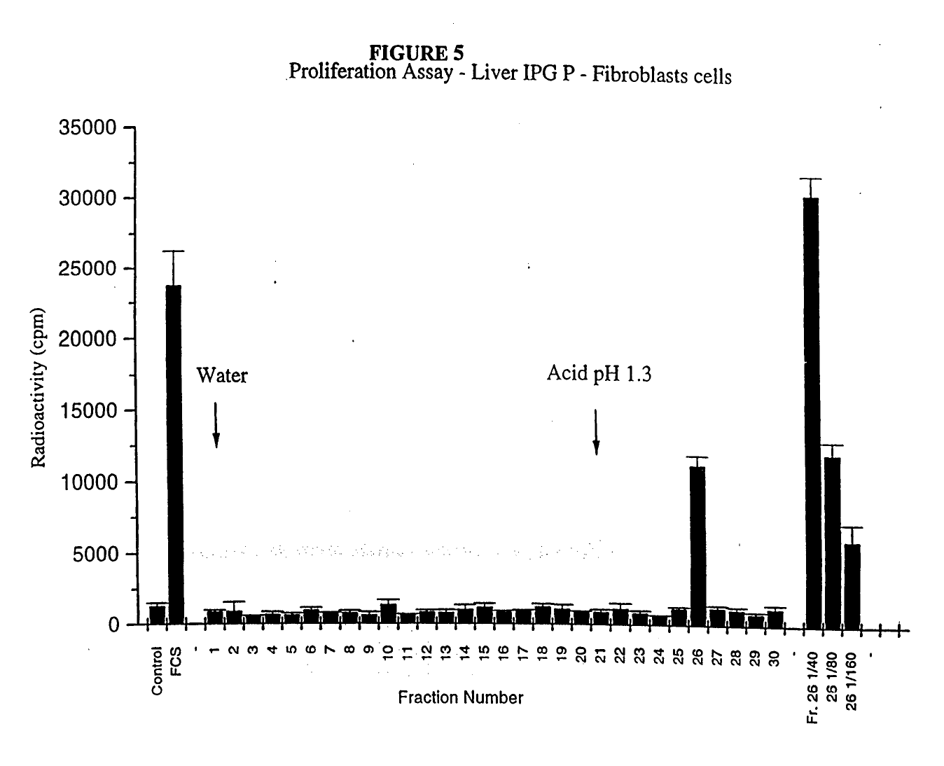

Test FaO cells were used to measure the stimulating activity of conditioned medium as described in methods, in the presence of the anti-IPG monoclonal antibodies 2D1, 5H6 and 2F1 at a concentration of 1 μg per well. The results in FIG. 4 are presented as radioactivity (cpm) obtained after incorporation of ³H-thymidine into DNA, for control (no serum addition), FCS (5% foetal calf serum+5% foetal bovine serum addition) and samples containing the antibodies. The results show that all three antibodies antagonise the proliferating activity caused by the addition of conditioned medium to the Fao cells, and that antibody 2F1 had the greatest inhibitory effect.

Rat Liver IPGs A and P Fractionation Profiles

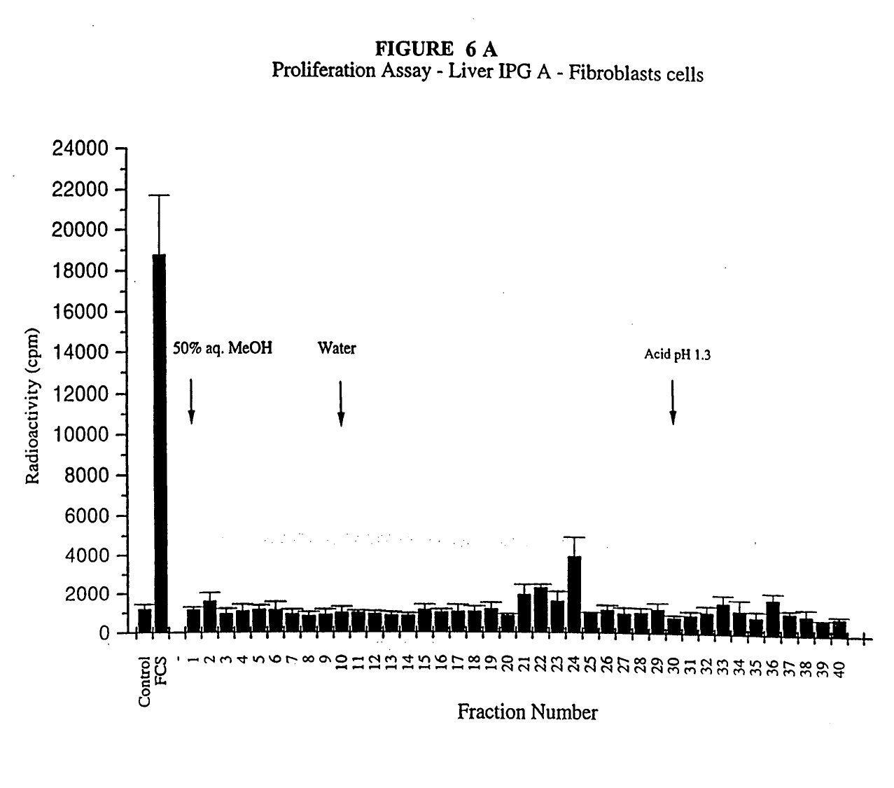

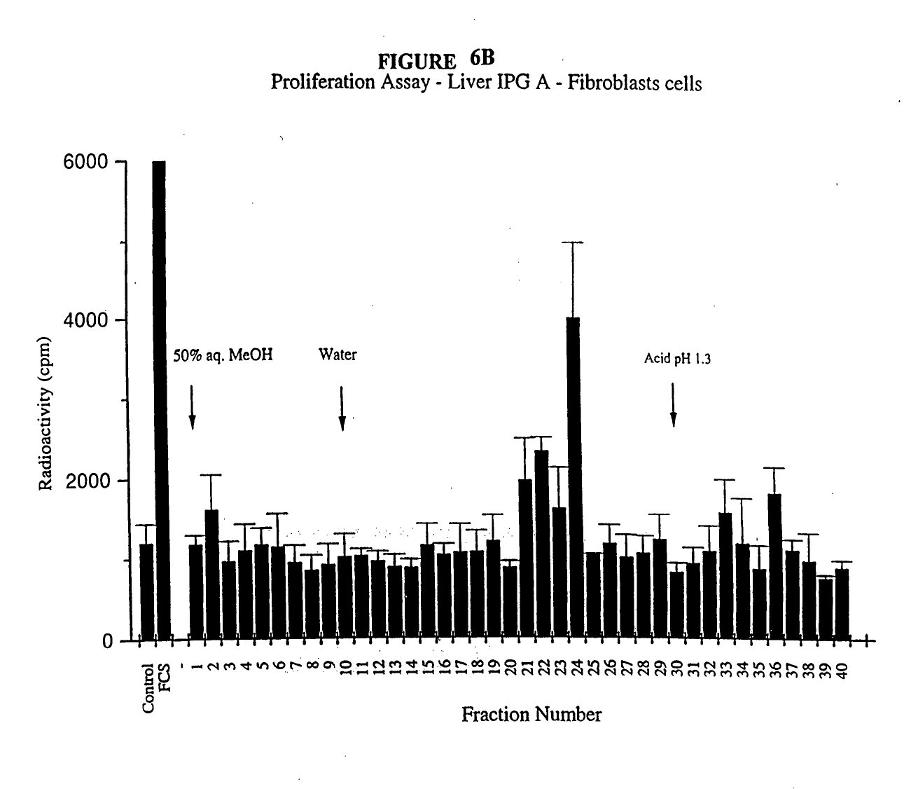

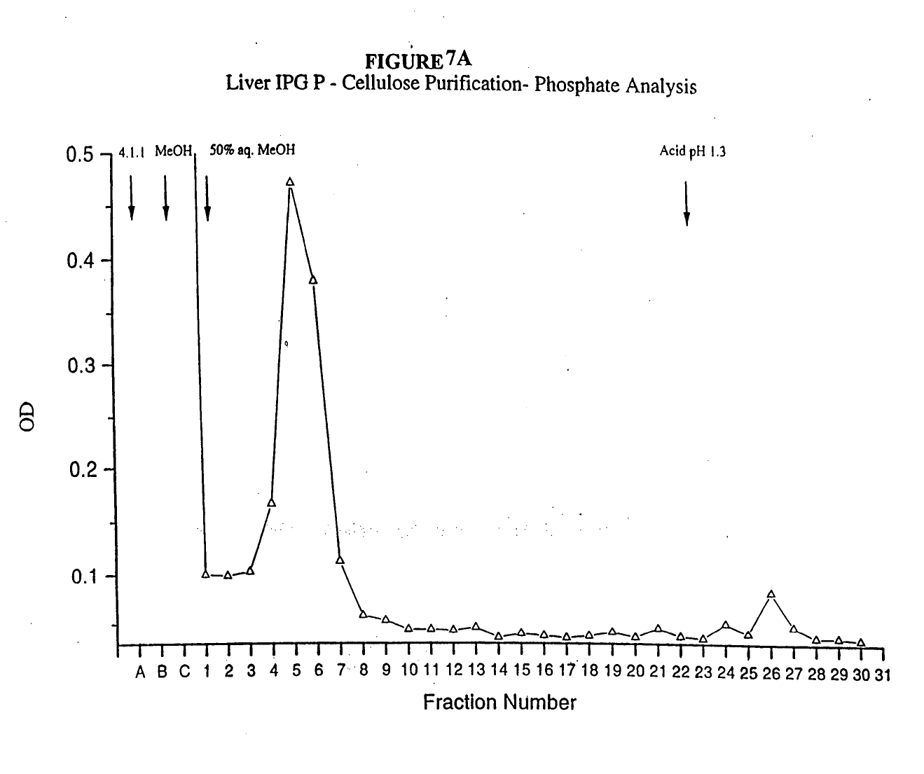

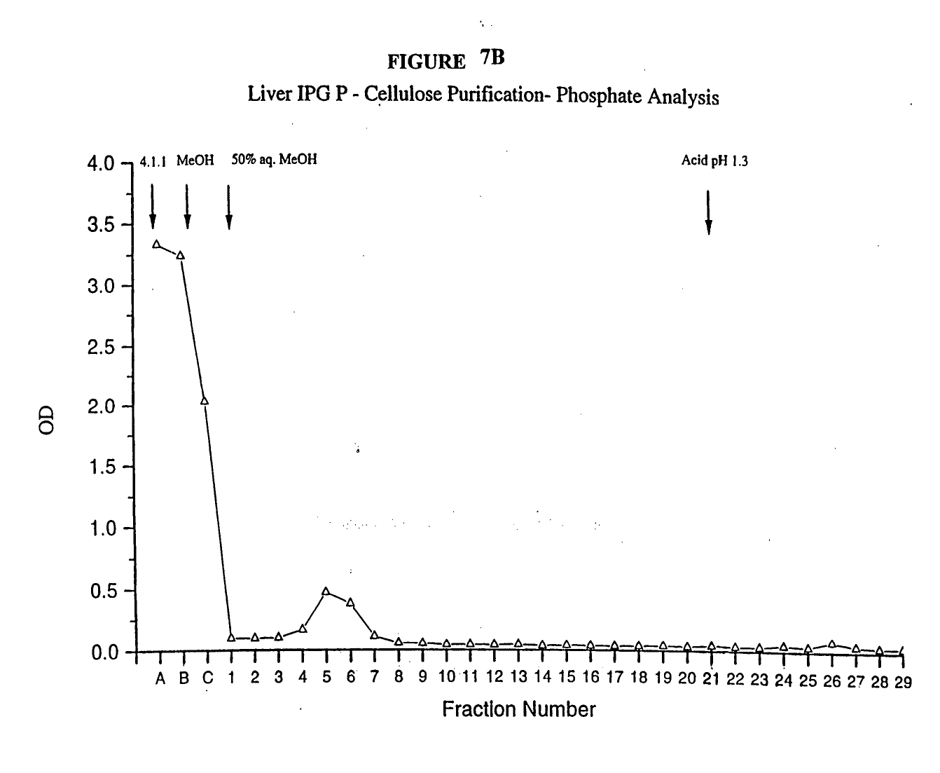

The IPGs obtained from two rat livers were fractionated using cellulose column chromatography as described in Methods. The fractions obtained were evaporated to dryness, redissolved in water (200 μl), and a portion of the solution (1.25 μl) assayed for biological activity in triplicates. The results are shown as radioactivity (cpm) obtained after incorporation of ³H-thymidine into DNA of 3T3EFGTR17 cells, for control (no serum addition), FCS (10% foetal calf addition), and fractions. All fractions were assayed at a final concentration of 1/80, except for fraction 26 of IPG-P (FIG. 5) which was also tested at a final concentration of 1/40 and 1/160. All fractions of the IPG-A (FIG. 6) were tested at a final concentration of 1/80. The arrows indicate the eluent system used at a particular fraction. The results indicate that cellulose chromatography is an effective technique for the isolation and purification of IPGs, e.g. IPGs obtained from natural source materials.

PDH Activity of Fraction 26 of IPG-P Type

PDH activity was measured as described in Caro et al, 1997. A portion (10 ml) of fractions 22, 26 and 27 was assayed. The result is presented as activation of the PDH complex expressed as units per gram of tissue. One unit of IPG PDH activity is the amount required to increase the basal rate of NADH production by 50%.
Fraction 22=0.123 units/g
Fraction 26=2.09 units/g
Fraction 27=0.523 units/g.

The results show that the IPGs purified from rat liver using cellulose chromatography have a characteristic P-type IPG biological activity.

Phosphate Analysis of Rat Liver IPG-P

The IPG-P obtained from two rat livers was fractionated by column chromatography using cellulose as described under Methods. Fractions (5 ml) were collected, concentrated to dryness, dissolved in water (200 μl) and a portion (100 μl) was assayed for phosphate as described in Caro et al, 1997. Each fraction was evaporated to dryness and hydrolized with perchloric acid (70% by volume) at 180° C. for 30 min. After cooling at room temperature, distilled water (250 μl) was added.

Ammonium molybdate and ascorbic acid were sequentially added, yielding final concentrations of 3 and 72 mM, respectively. Colour development was achieved by heating at 95° C. for 15 min. Optical absorbance was measured at 650 nm. The results in FIG. 8 are presented as OD obtained per fraction. Arrows indicate the eluent system used at a particular fraction. The results show that the P-type IPGs purified using cellulose chromatography contain phosphate.

Deposits

The deposit of hybridomas 2F7, 2D1 and 5H6 in support of this application was made at the European Collection of Cell Cultures (ECACC) under the Budapest Treaty by Rademacher Group Limited (RGL), The Windeyer Building, 46 Cleveland Street, London W1P 6DB, UK. The deposits have been accorded accession numbers accession numbers 98051201, 98031212 and 98030901 on 12 May 1998 ('201) and 9 Mar. 1998 ('212 and '901). RGL give their unreserved and irrevocable consent to the the materials being made available to the public in accordance with appropriate national laws governing the deposit of these materials, such as Rules 28 and 28a EPC. The expert solution under Rule 28(4) EPC is also hereby requested.

### REFERENCES

The references referred to herein are expressly incorporated by reference.
WO98/11116 and WO98/11117 (Rademacher Group Limited).
Rademacher et al, Brazilian J. Med. Biol. Res., 27:327-341, 1994.
Caro et al, Biochem. Mol. Med., 61:214-228, 1997.
Witters et al, J. Bio. Chem., 263:8027-8036, 1986.

1. Use of a substance which is an inositolphosphoglycan (IPG) antagonist having the property of reducing tumour cell proliferation for the preparation of a medicament for the treatment of cancer.

2. The use of claim 1, wherein the substance is an antagonist of an A-type substance which is a cyclitol containing carbohydrate and has the biological activity of causing tumour cell proliferation.

3. The use of claim 1 or claim 2, wherein the antagonist is:
(a) a substance which is capable of inhibiting the release of IPGs; or,
(b) a substance capable of reducing the levels of IPGs by binding to the IPGs; or,
(c) a substance which is a competitive agent which capable of reducing an effect of IPGs.

4. The use of claim 3, wherein the antagonist is a competitive IPG antagonist.

5. The use of claim 3, wherein the IPG antagonist is an anti-IPG antibody which is capable of specifically binding IPGs.

6. The use of claim 5, wherein the antibody capable of neutralising an activity of the IPGs.

7. The use of claim 6, wherein activity of the IPGs is the proliferation of tumour cells.

8. The use of any one of claims 5 to 7, wherein the antibody is a monoclonal antibody produced by hybridoma 2F7, 2D1 or 5H6, deposited at ECACC under accession numbers 98051201, 98031212 and 98030901.

9. The use of claim 3, wherein the antagonist is an inhibitor of glycosylphosphadtidylinositol specific phospholipase type C (GPI-PLC).

10. Use of the presence or amount of inositolphosphoglycans (IPGs) in a sample from a patient for the diagnosis and/or prognosis of cancer.

11. A method for the diagnosis and/or prognosis of cancer, the method comprising determining the presence or amount of inositolphosphoglycans in a sample from a patient.

12. The method of claim 11, wherein the presence or amount of the IPGs is determined by measuring a biological activity of an A-type substance.

13. The method of claim 12, wherein the biological activity of the A-type substance is inhibition of cAMP dependent protein kinase or causing tumour cell proliferation.

14. The method of claim any one of claims 11 to 13, wherein the method comprises the steps of:
(a) contacting a sample from a patient with a solid support having immobilised thereon a binding agent having binding sites which are capable of specifically binding to the IPGs with a sample from a patient under conditions in which the IPGs bind to the binding agent; and,
(b) determining the presence or amount of the IPGs bound to the binding agent.

15. The method of claim 14, wherein step (b) comprises (i) contacting the solid support with a developing agent which is capable of binding to occupied binding sites, unoccupied binding sites or the bound IPGs, the developing agent comprising a label and (ii) detecting the label to obtain a value representative of the presence or amount of the IPGs in the sample.

16. The method of claim 15, further comprising comparing the value with standards from healthy or cancerous tissues.

17. The method of any one of claims 14 to 16, wherein the label is a radioactive label, a chemiluminescent label, a fluorophor, a phosphor, a laser dye, a chromogenic dye, a macromolecular colloidal particle, a latex bead which is coloured, magnetic or paramagnetic, or an enzyme which catalyses a reaction producing a detectable result.

18. The method of any one of claims 14 to 17, wherein the binding agent immobilised on the solid support is an antibody which is capable of binding to the IPGs.

19. The method of any one of claims 14 to 18, wherein the binding agent is immobilised at a predefined location on the solid support.

20. Use of cellulose chromatography for purifying or isolating a P or A-type substance, wherein the substance is a cyclitol containing carbohydrate which is:
(i) a P-type substance having the biological activity of activating pyruvate dehydrogenase (PDH) phosphatase; or,
(ii) an A-type substance having the biological activity of inhibiting cAMP dependent protein kinase.

21. The use of claim 20, wherein the use involves contacting a sample containing P or A-type substance with a column containing cellulose and eluting the substance from the column.

22. The use of claim 20 or claim 21, wherein the column comprises microcrystalline cellulose.

23. A method of purifying or isolating a P or A-type substance, wherein the substance is a cyclitol containing carbohydrate which is:
(i) a P-type substance having the biological activity of activating pyruvate dehydrogenase (PDH) phosphatase; or,
(ii) an A-type substance having the biological activity of inhibiting cAMP dependent protein kinase;
wherein the method comprises:
(a) loading a column containing cellulose with a sample containing the P or A-type substance so that P or A-type substance binds to the column; and,
(b) eluting the P or A-type substance from the column.

24. The method of claim 23, wherein the cellulose is microcrystalline cellulose.

25. The method of claim 23 or claim 24, further comprising the step of dissolving the sample containing the P or A-type substance in 4/1/1 butanol/water/ethanol (B:W:E) prior loading on the column.

26. The method of any one of claims 23 to 25, further comprising the step of washing the column with B:W:E and methanol.
